(19) United States
(12) Patent Application Publication
(10) Pub. No.: US 2018/0125951 A1
(43) Pub. Date: May 10, 2018

(54) METHODS FOR ALLEVIATING HISTAMINE-INDEPENDENT PRURITUS USING NEUROTOXINS

(71) Applicant: Allergan, Inc., Irvine, CA (US)

(72) Inventors: Edward Hsia, Irvine, CA (US); Daniel Gil, Corona Del Mar, CA (US)

(21) Appl. No.: 15/808,402

(22) Filed: Nov. 9, 2017

Related U.S. Application Data
(60) Provisional application No. 62/420,377, filed on Nov. 10, 2016.
(60) Provisional application No. 62/510,044, filed on May 23, 2017.

(51) Int. Cl. A61K 38/48; A61P 17/04; A61P 17/10; A61P 17/08; A61P 17/16; A61P 17/06; A61P 17/00; A61K 9/00; A61K 45/06

(52) U.S. Cl. CPC A61K 38/4893; A61P 17/04; A61P 17/10; A61P 17/08; A61K 45/06; A61P 17/06; A61P 17/00; A61K 9/0019; A61K 9/0014; A61P 17/16

(57) ABSTRACT

Methods for treating, reducing the occurrence or preventing a histamine-independent pruritic itch using a clostridial toxin derivative are provided herein. In some aspects, the clostridial toxin derivative reduces the itch intensity or duration without affecting thermal or pain sensitivity.

### CROSS REFERENCE TO RELATED APPLICATIONS

This application claims the benefit of U.S. Provisional Application Ser. No. 62/420,377, filed Nov. 10, 2016, and 62/510,044, filed May 23, 2017, all incorporated entirely by reference.

### FIELD

The present disclosure relates to methods for treating pruritus. In particular, the present disclosure relates to methods for treating histamine-independent itch using neurotoxins

### BACKGROUND

Itch is defined as an unpleasant sensation that evokes a desire to scratch. In contrast to acute itch that is transient, chronic itch is a persistent, debilitating condition for which there are few treatment options. Chronic itch accompanies a number of skin diseases and systemic conditions, including eczema, kidney failure, liver cirrhosis and some cancers. A variety of neurological disorders also induce severe, chronic itch; for example, multiple sclerosis, diabetic neuropathy and post-herpetic neuralgia (shingles). Chronic itch, like chronic pain, can occur without injury or disease, serves no apparent biological purpose and has no recognizable endpoint.

Primary afferent sensory neurons detect a multitude of sensory stimuli, including touch, temperature and chemical mediators that cause itch. Itch-sensitive neurons are often classified according to their sensitivity to histamine, the best-studied pruritogen, or itch mediator.

Itch can be classified as histamine-dependent or histamine-independent. Antihistamine treatments are usually effective for histamine-dependent itch. Antihistamine treatments appear ineffective for histamine-independent itch. Most chronic pruritic diseases appear to be histamine-independent and are insensitive to antihistamine treatments.

Thus, there is a need for an effective treatment of histamine-independent itch.

### SUMMARY

In one aspect, the present disclosure provides a method for treating a histamine-independent itch, comprising locally administering a composition comprising a therapeutically effective amount of a clostridial toxin derivative to an itch affected area. In some embodiments, the clostridial toxin derivative is a native or recombinant neurotoxin, a recombinant modified toxin, fragments thereof, or combinations thereof. In one embodiment, the present method comprises locally administering a clostridial toxin derivative into an itch affected area. In one embodiment, the clostridial toxin derivative is a botulinum toxin. In one embodiment, the clostridial toxin derivative is a botulinum toxin type A.

In another aspect, the present disclosure provides a method for alleviating a symptom associated with histamine-independent itch treating a histamine-independent itch, comprising locally administering a composition comprising a therapeutically effective amount of a clostridial toxin derivative to an itch affected area. In some embodiment, the symptom associated with histamine independent itch comprises pain, discomfort, inflammation, or combinations thereof. In some embodiments, the administering reduces the itch intensity and/or the itch duration. In some embodiments, the administering reduces the itch intensity and/or the itch duration without affecting thermal sensitivity or pain thresholds. In another aspect, by alleviating one or more symptoms associated with chronic itch in a patient in need thereof, the present method provides functional improvement and thus improves the quality of life for the patient.

In another aspect, the present disclosure provides a method for reducing the occurrence of a histamine-independent itch, comprising locally administering a composition comprising a therapeutically effective amount of a clostridial toxin derivative to an itch affected area. In some embodiments, the administering reduces the itch intensity and/or the itch duration. In some embodiments, the administering reduces the itch intensity and/or the itch duration without affecting thermal sensitivity or pain thresholds. In some embodiments, the clostridial toxin derivative is a native or recombinant neurotoxin, a recombinant modified toxin, fragments thereof, or combinations thereof. In one embodiment, the present method comprises locally administering a clostridial toxin derivative into an itch affected area. In one embodiment, the clostridial toxin derivative is a botulinum toxin. In one embodiment, the clostridial toxin derivative is a botulinum toxin type A.

### BRIEF DESCRIPTION OF THE DRAWINGS

The following drawings are presented to illustrate aspects and features of embodiments of the present invention.

FIG. 1. Use of an exemplary clostridial toxin derivative to treat a cowhage-induced itch and pain according to aspects of the present method. On one arm, a designated area was treated by the exemplary clostridial toxin derivative; on the other arm, a second designated area was treated by a control (vehicle or saline). Each area was submitted to itch and pain testing;

FIGS. 2A-2E. Measurement of cowhage-induced itch intensity (VAS) over a 10-minute period at various timepoints (baseline (FIG. 2B); 1-week (FIG. 2C), 1-month (FIG. 2D), and 3-months (FIG. 2E)) following intradermal injection of the exemplary clostridial toxin derivative or saline; FIG. 2A is a summary graph at all time points

FIGS. 3A-3D. Measurement of peak itch intensity (VAS) (FIGS. 3A and 3B) and duration (FIGS. 3C and 3D) of cowhage-induced itch at various timepoints (1-week, 1-month, and 3-months) following intradermal injection of the exemplary clostridial toxin derivative or saline;

FIG. 4. Percent change in warm threshold at various timepoints (1-week, 1-month, and 3-months) following intradermal injection of the exemplary clostridial toxin derivative or saline; and

FIG. 5. Percent change in pain threshold at various timepoints (1-week, 1-month, and 3-months) following intradermal injection of the exemplary clostridial toxin derivative or saline.

### DETAILED DESCRIPTION OF THE PREFERRED EMBODIMENTS

Botulinum neurotoxins (BoNTs) such as, for example, BoNT/A, BoNT/B, etc., act on the nervous system by blocking the release of neurosecretory substances such as neurotransmitters. The action of BoNT is initiated by its binding to a receptor molecule on the cell surface, and then the toxin-receptor complex undergoes endocytosis. Once inside the cell, BoNT cleaves exocytotic specific proteins responsible for neurotransmitter docking and release from the cell known as the SNARE proteins (soluble N-ethylmaleimide-sensitive factor attachment protein receptor). The resulting transient chemodenervation has been utilized medically to block motor neurotransmission at the neuromuscular junction leading to a variety of therapeutic applications.

The following definitions apply herein:

“About” or “approximately” as used herein means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value.

“Administration”, or “to administer” means the step of giving (i.e. administering) a botulinum toxin to a subject, or alternatively a subject receiving a pharmaceutical composition. The present method can be performed via administration routes including intramuscular, non-intramuscular, intradermal, subcutaneous administration, intrathecal administration, intraperitoneal administration, implantation (for example, of a slow-release device such as polymeric implant or miniosmotic pump), instillation, or combinations thereof.

“Alleviating” means a reduction in the occurrence of a pain or other symptoms associated with pruritic itch. Thus, alleviating includes some reduction, significant reduction, near total reduction, and total reduction. An alleviating effect may not appear clinically for between 1 to 7 days after administration of a clostridial toxin derivative to a patient or sometimes thereafter.

“Botulinum toxin” means a neurotoxin produced by Clostridium botulinum, as well as a botulinum toxin (or the light chain or the heavy chain thereof) made recombinantly by a non-Clostridial species. The term “botulinum toxin”, as used herein, encompasses the botulinum toxin serotypes A, B, C, D, E, F and G, and their subtypes and any other types of subtypes thereof, or any re-engineered proteins, analogs, derivatives, homologs, parts, sub-parts, variants, or versions, in each case, of any of the foregoing. “Botulinum toxin”, as used herein, also encompasses a “modified botulinum toxin”. Further “botulinum toxin” as used herein also encompasses a botulinum toxin complex, (for example, the 300, 600 and 900 kDa complexes), as well as the neurotoxic component of the botulinum toxin (150 kDa) that is unassociated with the complex proteins.

“Clostridial toxin derivative” refers to a molecule which contains any part of a clostridial toxin. As used herein, the term “clostridial toxin derivative” encompasses native or recombinant neurotoxins, recombinant modified toxins, fragments thereof, or combinations thereof.

“Clostridial toxin” refers to any toxin produced by a Clostridial toxin strain that can execute the overall cellular mechanism whereby a Clostridial toxin intoxicates a cell and encompasses the binding of a Clostridial toxin to a low or high affinity Clostridial toxin receptor, the internalization of the toxin/receptor complex, the translocation of the Clostridial toxin light chain into the cytoplasm and the enzymatic modification of a Clostridial toxin substrate.

“Effective amount” as applied to the biologically active ingredient means that amount of the ingredient which is generally sufficient to induce a desired change in the subject. For example, where the desired effect is a reduction in intensity or occurrence of pruritic itch, an effective amount of the ingredient is that amount which causes at least a substantial reduction of pruritic itch and associated symptoms, and without resulting in significant toxicity.

“Implant” means a controlled release (e.g., pulsatile or continuous) composition or drug delivery system. The implant can be, for example, injected, inserted or implanted into a human body.

“Local administration” means administration of a pharmaceutical agent to or to the vicinity of a muscle or a subdermal location in a patient by a non-systemic route. Thus, local administration excludes systemic routes of administration, such as intravenous or oral administration.

“Peripheral administration” means administration to a location away from a symptomatic location, as opposed to a local administration.

“Treating” or “treatment” means to alleviate (or to eliminate) at least one symptom (such as, for example, hip and groin pain), either temporarily or permanently.

“Therapeutically effective amount” refers to an amount sufficient to achieve a desired therapeutic effect.

Aspects of the present disclosure provide in part a method for treating, reducing the occurrence or preventing pruritus, including histamine-independent pruritic itch. In another aspect, the present disclosure provides a method for alleviating one or more symptoms associated with pruritus, including histamine-independent pruritic itch.

In one aspect, the present disclosure provides a method for treating a histamine-independent pruritic itch in a patient in need thereof, the method comprising locally administering a composition comprising a therapeutically effective amount of a clostridial toxin derivative to an itch affected area. In some embodiments, the administering reduces the itch intensity and/or the itch duration. In some embodiments, the administering reduces the itch intensity and/or duration without affecting the thermal sensitivity and/or pain threshold in the patient.

In some embodiments, the composition is administered by injections, including intramuscular injections or non-intramuscular injections. In alternative embodiments, the composition is administered topically.

In some embodiments, the clostridial toxin derivative includes a native, recombinant clostridial toxin, recombinant modified toxin, fragments thereof, or combinations thereof. In some embodiments, the clostridial toxin derivative is a botulinum toxin. In some embodiments, the botulinum toxin can be a botulinum toxin type A, type B, type C₁, type D, type E, type F, or type G, or any combination thereof. The botulinum neurotoxin can be a recombinantly made botulinum neurotoxins, such as botulinum toxins produced by E. coli.

In some embodiments, the botulinum neurotoxin can be a modified neurotoxin, that is a botulinum neurotoxin which has at least one of its amino acids deleted, modified or replaced, as compared to a native toxin, or the modified botulinum neurotoxin can be a recombinant produced botulinum neurotoxin or a derivative or fragment thereof. In certain embodiments, the modified toxin has an altered cell targeting capability for a neuronal or non-neuronal cell of interest. This altered capability is achieved by replacing the naturally-occurring targeting domain of a botulinum toxin with a targeting domain showing a selective binding activity for a non-botulinum toxin receptor present in a non-botulinum toxin target cell. Such modifications to a targeting domain result in a modified toxin that can selectively bind to a non-botulinum toxin receptor (target receptor) present on a non-botulinum toxin target cell (re-targeted). A modified botulinum toxin with a targeting activity for a non-botulinum toxin target cell can bind to a receptor present on the non-botulinum toxin target cell, translocate into the cytoplasm, and exert its proteolytic effect on the SNARE complex of the target cell. In essence, a botulinum toxin light chain comprising an enzymatic domain is intracellularly delivered to any desired cell by selecting the appropriate targeting domain.

The clostridial toxin derivative, such as a botulinum toxin, for use according to the present invention can be stored in lyophilized, vacuum dried form in containers under vacuum pressure or as stable liquids. Prior to lyophilization the botulinum toxin can be combined with pharmaceutically acceptable excipients, stabilizers and/or carriers, such as, for example, albumin, or the like. In embodiments containing albumin, the albumin can be, for example, human serum albumin, or the like. The lyophilized material can be reconstituted with a suitable liquid such as, for example, saline, water, or the like to create a solution or composition containing the botulinum toxin to be administered to the patient.

In some embodiments, the clostridial toxin derivative is provided in a controlled release system comprising a polymeric matrix encapsulating the clostridial toxin derivative, wherein fractional amount of the clostridial toxin derivative is released from the polymeric matrix over a prolonged period of time in a controlled manner. Controlled release neurotoxin systems have been disclosed for example in U.S. Pat. Nos. 6,585,993; 6,585,993; 6,306,423 and 6,312,708, each of which is hereby incorporated by reference in its entirety.

The therapeutically effective amount of the clostridial toxin derivative, for example a botulinum toxin, administered according to the present method can vary according to the potency of the toxin and particular characteristics of the pain being treated, including its severity and other various patient variables including size, weight, age, and responsiveness to therapy. The potency of the toxin is expressed as a multiple of the LD₅₀ value for the mouse, one unit (U) of toxin being defined as being the equivalent amount of toxin that kills 50% of a group of 18 to 20 female Swiss-Webster mice, weighing about 20 grams each.

The therapeutically effective amount of the botulinum toxin, in the present method can vary according to the potency of a particular botulinum toxin, as commercially available Botulinum toxin formulations do not have equivalent potency units. For example, one unit of BOTOX® (onabotulinumA), a botulinum toxin type A available from Allergan, Inc., has a potency unit that is approximately equal to 3 to 5 units of DYSPORT® (abobotulinumA), also a botulinum toxin type A available from Ipsen Pharmaceuticals. MYOBLOC®, a botulinum toxin type B available from Elan, has a much lower potency unit relative to BOTOX®. In some embodiments, the botulinum neurotoxin can be a pure toxin, devoid of complexing proteins, such as XEOMIN® (incobotulinumtoxinA). One unit of IncobotulinumtoxinA has a potency approximately equivalent to one unit of onabotulinumtoxinA. Thus, the quantity of toxin administered and the frequency of its administration will be at the discretion of the physician responsible for the treatment and will be commensurate with questions of safety and the effects produced by a particular toxin formulation.

The dosages used in human therapeutic applications are roughly proportional to the mass of the tissue being injected. Typically, the dose of a clostridial toxin derivative administered to the patient may be up from about 0.01 to about 1,000 units; for example, up to about 500 units, and preferably in the range from about 10 to about 460 units per patient per treatment, although smaller of larger doses may be administered in appropriate circumstances.

In some embodiments, the present method comprises administering a composition comprising about 10-500 units of a botulinum toxin type A, such as BOTOX®, into an itch affected area. In some embodiments, the present method comprises administering a composition comprising about 10-300 units of BOTOX® into the itch affected area. In one specific embodiment, the present method comprises administering a composition comprising about 10-500 units of BOTOX® by injection into the itch affected area (1-2 units per cm² by intradermal injection). In some embodiments, the composition is administered topically. In certain embodiments, the dosage can range from about 10 Units to about 200 U per treatment. In some embodiments, the pharmaceutical composition can be administered at multiple sites, ranging from 1 site up to about 50 sites. In some embodiments, if the neurotoxin is botulinum toxin type B, the dosage is approximately 50 times greater than the functionally equivalent dosage of botulinum toxin type A.

In some embodiments, the clostridial toxin derivative is co-administered with an antihistamine treatment. In other embodiments, the clostridial toxin derivative is co-administered with any therapies suitable for pruritus treatment, including but not limited to therapies using opioids, NK1 antagonists, TRPV antagonists, TRPA antagonists PAR2 antagonists, IL-31 antagonists, TSLP antagonists as well as therapeutic agents used to treat skin diseases. Exemplary therapeutic agents suitable for treatment of skin disease include but are not limited to steroids, calcineurin inhibitors, anti-TNF, anti-IL-17, anti-IL-23, anti-IL4R treatments, retinoids, vitD analogs, PDE4 inhibitors, or combinations thereof.

In some embodiments, the anti-histamine treatment is administered simultaneously with the clostridial toxin derivative. In alternative embodiments, the antihistamine treatments is administered sequentially relative to the clostridial toxin derivative.

In some embodiments, the pruritus treatment or skin therapeutic agent is administered simultaneously with the clostridial toxin derivative. In alternative embodiments, the pruritus treatment or skin therapeutic agent is administered sequentially relative to the clostridial toxin derivative.

The treatment effects of the clostridial toxin derivative can persist for between about 1 month and 5 years. Administration can be repeated as necessary. As a general guideline, botulinum toxin type A administered into or near muscle tissue has been observed to produce flaccid paralysis at target site muscles for up to about 3 to 6 months. However, increased efficacy of the treatment using botulinum toxin type A is expected to happen when the toxin is administered according to the disclosed method at about 3 month intervals. Repeated administration of the clostridial toxin derivative according to aspects of the present method reduces the occurrence or prevents chronic pruritic diseases associated itch.

Thus, in another aspect, the present disclosure provides a method for reducing the occurrence or preventing histamine-independent pruritic itch in a patient in need thereof, the method comprising locally administering a composition comprising a therapeutically effective amount of a clostridial toxin derivative into an itch affected area. In some embodiments, the administering reduces the itch intensity and/or the itch duration. In some embodiments, the administering reduces the itch intensity and/or duration without affecting the thermal sensitivity and/or pain threshold in the patient.

In yet another aspect, the present disclosure provides a method for alleviating the pain, discomfort and inflammation due to itch associated with histamine-independent pruritic itch in a patient in need thereof, the method comprising locally administering a composition comprising a therapeutically effective amount of a clostridial toxin derivative into an itch affected area. In some embodiments, the administering reduces the itch intensity and/or the itch duration. In some embodiments, the administering reduces the itch intensity and/or duration without affecting the thermal sensitivity and/or pain threshold in the patient.

In some embodiments, administration of a clostridial toxin derivative alleviates the itch sensation and/or duration without affecting the thermal sensitivity, the pain sensation, or combination thereof. In this respect, the clostridial toxin derivative of the present method does not act like an anesthetic. In alternative embodiments, administration of a clostridial toxin derivative alleviates itch, pain, thermal sensitivity, or combinations thereof.

According to the present disclosure, pruritus includes any itchy or pruritic condition, e.g., a sensation that causes the desire or reflex to scratch. In some embodiments, methods of the present invention are used for the treatment of a subject suffering from a pruritic condition selected from the group consisting of atopic dermatitis, nervous dermatitis, contact dermatitis, seborrheic dermatitis, autosensitization dermatitis, caterpillar dermatitis, asteatosis, senile pruritus cutaneous, insect sting, photosensitive dermatosis, urticarial, prurigo, herpes, impetigo, eczema, tinea, lichen, psoriasis, scabies and acne vulgaris, visceral diseases complicated with pruritus such as malignant tumors, diabetes mellitus, hepatic diseases, renal failure, hemodialysis, peritoneal dialysis, and pregnancy.

In some embodiments, the methods disclosed herein are used for the treatment of a subject suffering from a pruritic condition associated with a skin change. For example, such pruritic condition can be selected from the group consisting of pruritus associated with inflamed skin (e.g., atopic dermatitis, psoriasis, burns); pruritus arising from conditions not directly related to the skin (e.g., uremic pruritus, cholestatic pruritus, cancers, hydroxyethyl starch induced pruritus), and pruritus associated with chronic secondary scratch or other types of skin lesions that may or may not be the result of an underlying medical condition (e.g., prurigo nodularis) and the underlying disease is categorized based on histological, radiological or other investigations as being of an origin selected from the group consisting of dermatologic origin, systemic disease origin, neurologic origin, psychogenic origin, mixed origin, or other origin.

In some embodiments, the present methods are used for the treatment of a subject suffering from a pruritic condition associated with neurogenic inflammation of the skin, e.g., prurigo nodularis, atopic dermatitis, burn pruritus, burn, wound healing, etc. In some other embodiments, methods of the present invention are used for the treatment of a subject suffering from a pruritic condition associated with neurogenic inflammation with elevated substance P level. In still some other embodiments, methods of the present invention are used for the treatment of a subject suffering from a pruritic condition associated with elevated substance P level.

In some embodiments, the present methods are used for the treatment of a subject suffering from a pruritic condition associated with one or more related or unrelated conditions. For example, the pruritic condition can be associated with a dermatologic condition including aquagenic pruritus, atopic dermatitis, idiopathic pruritus, Lichen simplex chronicus, prurigo nodularis, psoriasis, and scabies. In another example, the pruritic condition can be associated with a hematological or oncological condition including cancer related pruritus, chemotherapy induced pruritus, HIV protease inhibitor induced pruritus, Hodgkin's lymphoma associated pruritus, polycythemia vera, etc. In another example, the pruritic condition can be associated with a metabolic condition including cholestatic pruritus, uremic pruritus, etc. In still another example, the pruritic condition can be associated with a condition of pain or neurological condition including brachioradial pruritus, burn induced pruritus, neuropathic pruritus, morphine induced pruritus, multiple sclerosis associated pruritus, post herpetic pruritus, pruritus associated with psychiatric causes, etc.

In one embodiment, the methods disclosed herein are used for the treatment of uremic pruritus. In another embodiment, the present methods are used for the treatment of prurigo nodularis. In yet another embodiment, the present methods are used to treat human beings. In still another embodiment, methods of the present invention are used to treat animals other than human beings.

A method within the scope of the present disclosure can provide improved patient function. “Improved patient function” can be defined as an improvement measured by factors such as a reduced pain, increased ambulation, healthier attitude, more varied lifestyle and/or healing permitted by normal muscle tone and function. Improved patient function is may be measured with an improved quality of life (QOL) or Health-Related Quality of Life (HRQL). Scores obtained can be compared to published values available for various general and patient populations.

The following non-limiting examples provide those of ordinary skill in the art with specific preferred methods to treat histamine-independent itch associated with pruritic diseases within the scope of the present disclosure, and it is not intended to limit the scope of the invention. In the following examples, various modes of non-systemic administration of a botulinum neurotoxin can be carried out. For example, by intramuscular injection, non-intramuscular injection, topically or by implantation of a controlled release implant.

### EXAMPLES

The following non-limiting examples provide those of ordinary skill in the art with specific preferred methods to treat conditions within the scope of embodiments of the present invention and are not intended to limit the scope of the invention.

### Example 1

A study was carried out to evaluate the antipruritic effect of an exemplary clostridial toxin derivative using a histamine-independent itch model (cowhage). Cowhage-induced itch may be more representative of chronic pruritic diseases compared to histamine-induced itch.

Briefly, 50 healthy subjects were recruited from which 30 subjects with no skin and systemic diseases were enrolled in the study. There were an equal number of male and female subjects between the ages of 18 and 50. The antipruritic effect of a botulinum toxin type A, BOTOX® (onabotulinumtoxinA), was evaluated by using cowhage for itch induction. The effect of BOTOX® on heat pain was also tested. Subjects underwent a follow up period testing the lasting effects of BOTOX® on itch and heat pain.

Procedures:

Forearm Test Sites

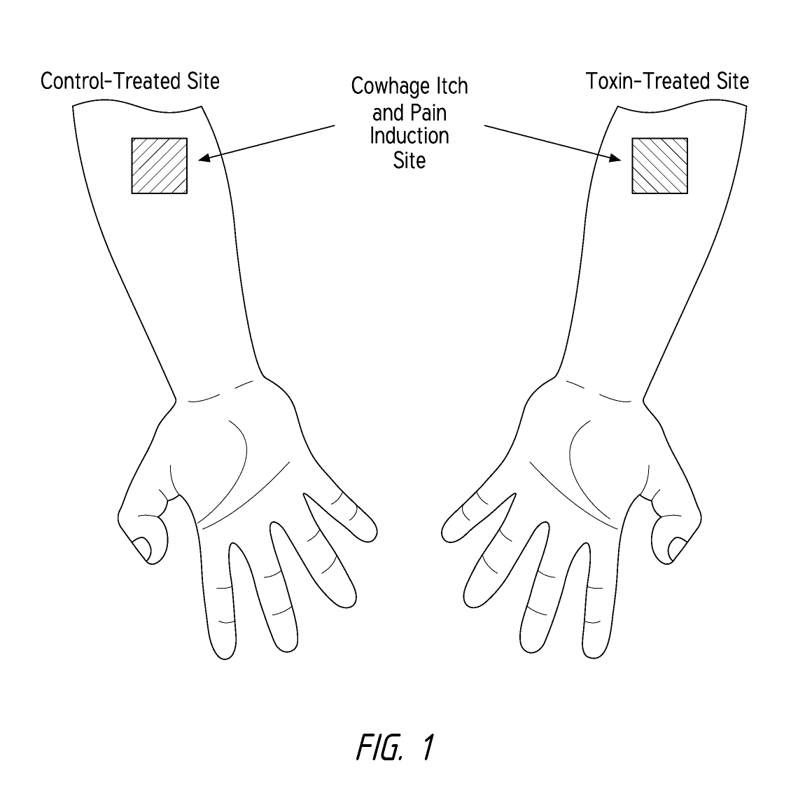

Two 4 cm×4 cm square areas were used on the subject's volar forearms (FIG. 1). On one of the subject's arms, a one 4 cm×4 cm area was injected with Botox® during the baseline visit (10 Units total divided into 5 intradermal injections). On the subject's contralateral arm, a one 4 cm×4 cm area was injected with a control (vehicle or saline).

The location (right or left arm) of treatment site and control sites was randomized and the subjects were blinded to the identity of the injections. These site locations were then measured and recorded.

Warmth (Thermal Sensitivity) and Heat Pain Thresholds and Heat Pain Intensity:

Thermal stimuli were delivered using the TSA-II Neurosensory Analyzer (Medoc Ltd, Ramat-Yishai, Israel) at the designated area, (each 4 cm×4 cm). The thermode (probe) warmed the skin surface at a linear rate of 0.4° C. s⁻¹ to a maximum of 50° C., from a baseline of 32° C. At 50° C. the stimulus automatically terminated. Warmth sensation thresholds and then heat pain detection thresholds were determined by the ascending method of limits three times. The subjects were instructed to respond on detection of a thermal stimulus. These values were subsequently used to compute the mean threshold.

Heat pain was induced by the thermal stimulation delivered with rise and fall rates of 6° C./s with a plateau duration of 5 seconds at 49° C. and a minimum interval of 30 seconds between stimuli at baseline (35° C.). Ratings of pain intensity were taken during the plateau temperature on a VAS (0-10) scale.

Itch Induction:

The 4 cm×4 cm test areas on the forearm was subjected to itch induction with cowhage after a 10-minute rest from the thermal testing. Ratings of itch intensity were taken on a VAS scale (0-10) every 30 seconds until itch subsides. Subjects were asked to not scratch the areas.

Cowhage itch was induced by the application of cowhage spicules on the skin. Cowhage or the velvet bean (Mucuna pruriens var. pruriens) is a tropical plant whose pods are covered with short, fine hairs (also called trichomes or “spicules”). A number of 40 to 45 cowhage spicules were applied to one area of the left inner forearm. The spicules were gently rubbed for 30-45 seconds onto the skin with a circular motion to facilitate contact until a substantial itch sensation was induced. After the itch sensation was gone, the cowhage spicules were removed using adhesive tapes.

Visual Analogue Scale and Numerical Scale

Healthy subjects were asked to measure their perception of warm sensation, heat pain, and itching with a 100 mm visual analogue scale (VAS) and an 11 number numerical scale 0-10 to report the perceived itch or pain intensity. VAS is anchored with the verbal descriptors of “no sensation” on the left and “the most intense, unbearable sensation imaginable” on the right.

Study Visits

### Visit 1:

1. Baseline thermal thresholding and pain VAS intensity at each site.
2. 10-minute rest.
3. Baseline itch VAS intensity at each site.
4. Injection of Botox and control (vehicle or saline) into the designate sites.

### Visit 2: (1 Week Follow Up)

1. Thermal thresholding and pain VAS intensity at each site.
2. 10-minute rest.
3. Itch VAS intensity at each site.

### Visit 3: (1 Month Follow Up)

1. Thermal thresholding and pain VAS intensity at each site.
2. 10-minute rest.
3. Itch VAS intensity at each site.

### Visit 4: (3 Month Follow Up)

1. Thermal thresholding and pain VAS intensity at each site.
2. 10-minute rest.
3. Itch VAS intensity at each site.
4. Subject Compensation.

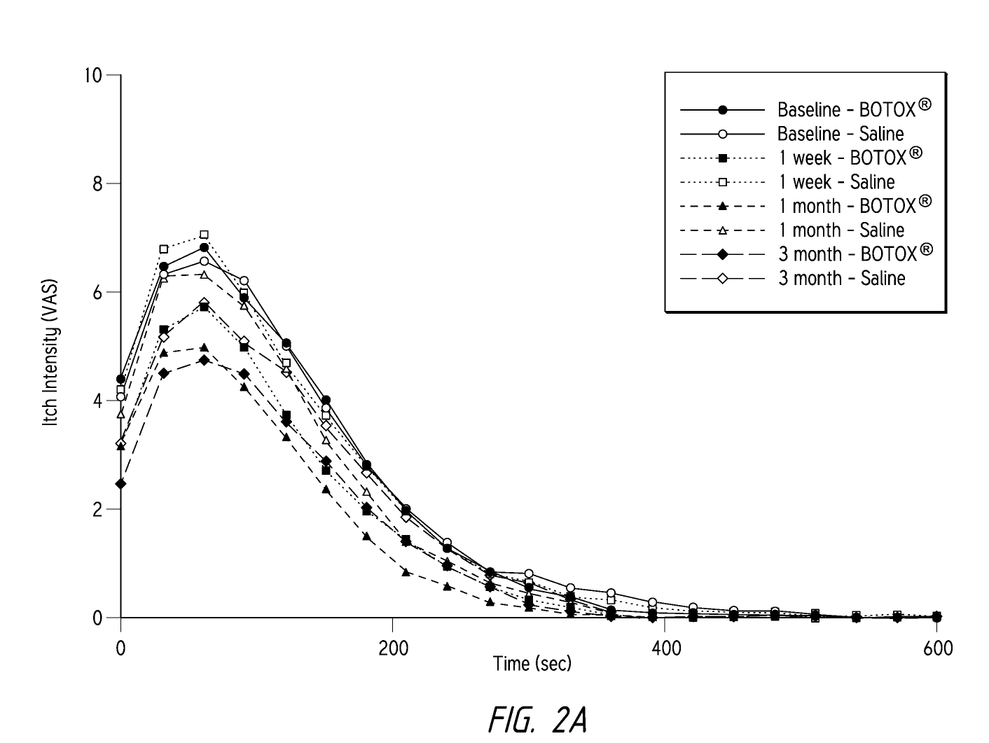

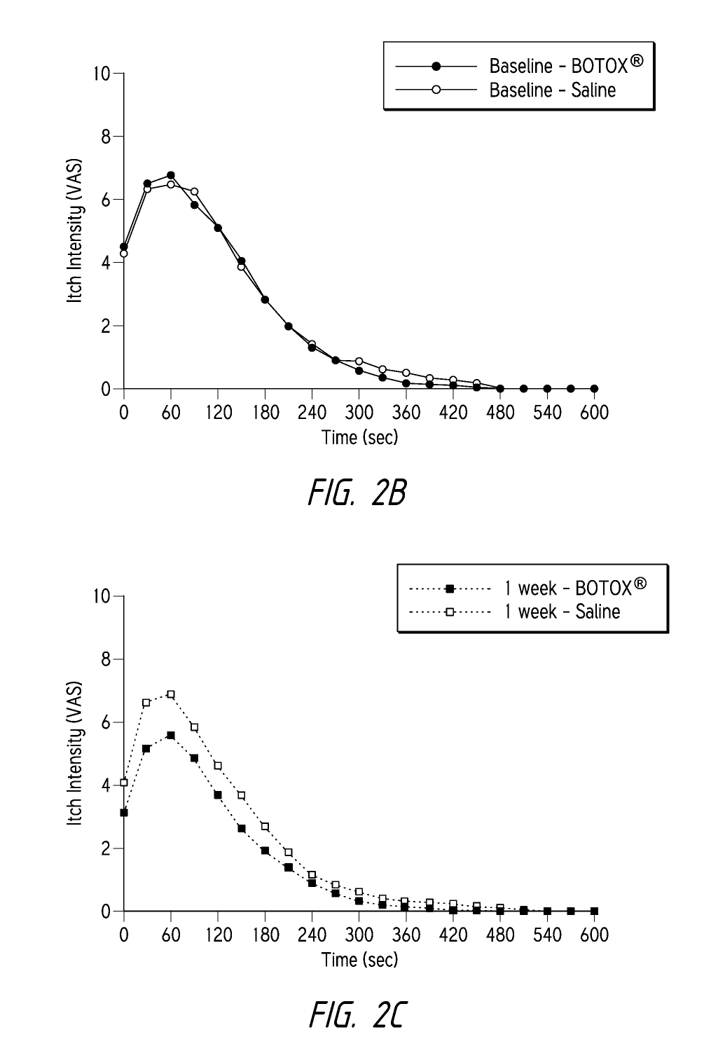

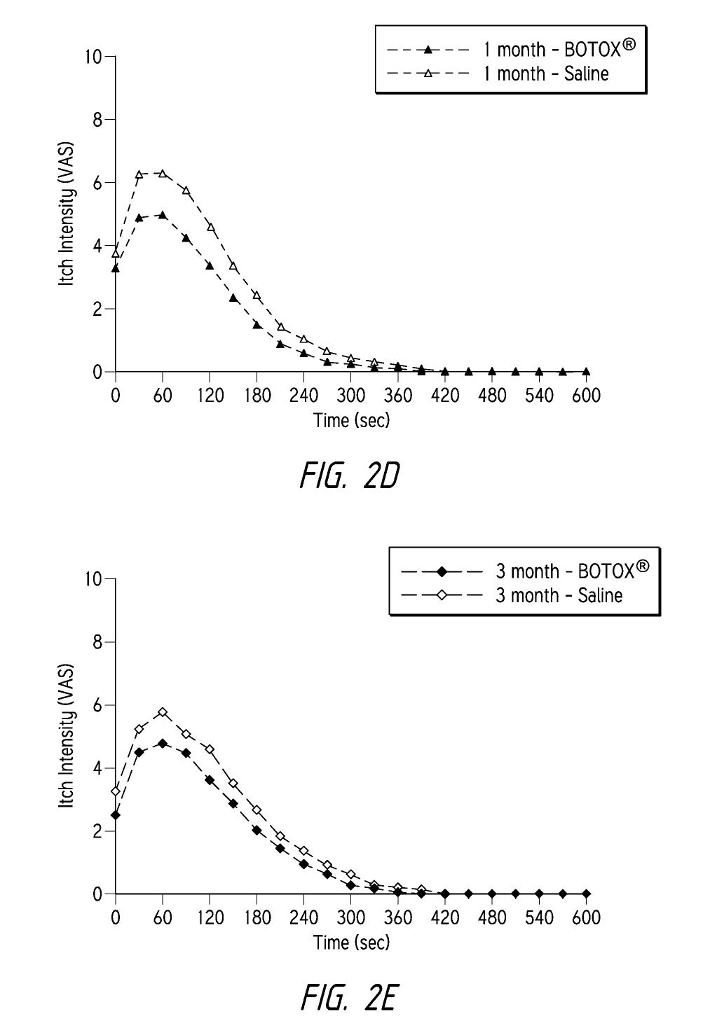

FIGS. 2A-2E show the time course of cowhage-induced itch intensity (VAS—0 to 10) over a 10-minute period at baseline and following treatment of a representative clostridial toxin, BOTOX®, or saline control. FIG. 2A is a summary of the itch intensity times courses of the saline control site and the toxin-treated site. FIGS. 2B-2E displays pairwise comparison between the saline control site and the toxin-treated site at each time point to facilitate comparison. As shown in FIG. 2B, at baseline, before any treatment was administered, the itch intensity time course did not significantly differ between the control-treated site and the toxin-treated site. At one week (FIG. 2C), one month (FIG. 2D), and 3 months (FIG. 2E) after treatment, BOTOX® significantly reduced itch intensity when compared to the saline control. FIGS. 2A-2E show that BOTOX® caused a significant reduction in itch VAS intensity for up to 3 months' post-treatment.

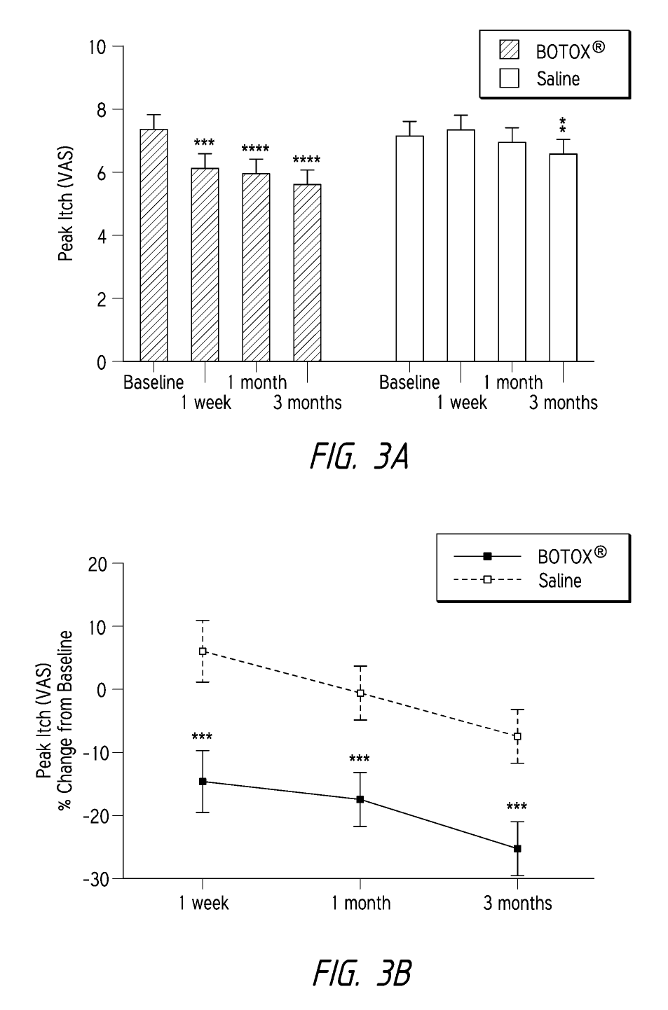

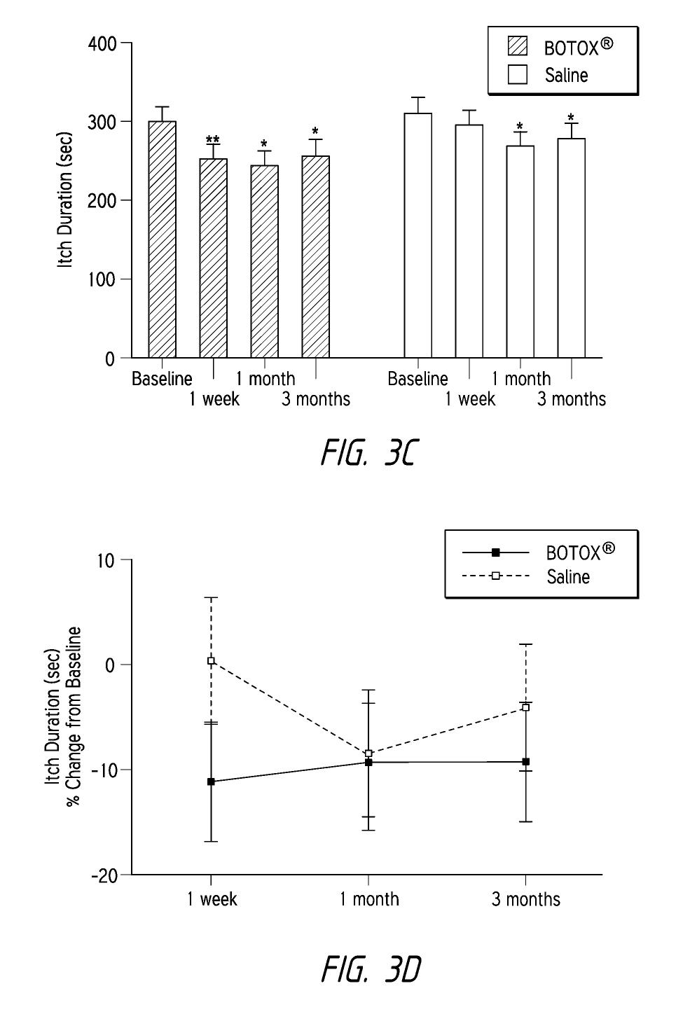

FIGS. 3A and 3B show the itch intensity; FIGS. 3C and 3D show the duration of the cowhage-induce itch following administration of BOTOX® or saline. Referring to FIGS. 3A and 3B, compared to baseline, BOTOX® treatment significantly decreased the itch intensity perceived at 1 week (p=0.001), 1 month (p<0.0001) and 3 months (p<0.0001) after treatment. Referring to FIGS. 3C and 3D, compared to baseline, the duration of the itch was significantly shortened by the BOTOX® treatment after 1 week (p=0.009), 1 month (p=0.027) and 3 months (p=0.015). The asterisks refer to statistical significance, where *=p<0.05, **=p<0.01, ***=p<0.001, ****=p<0.0001.

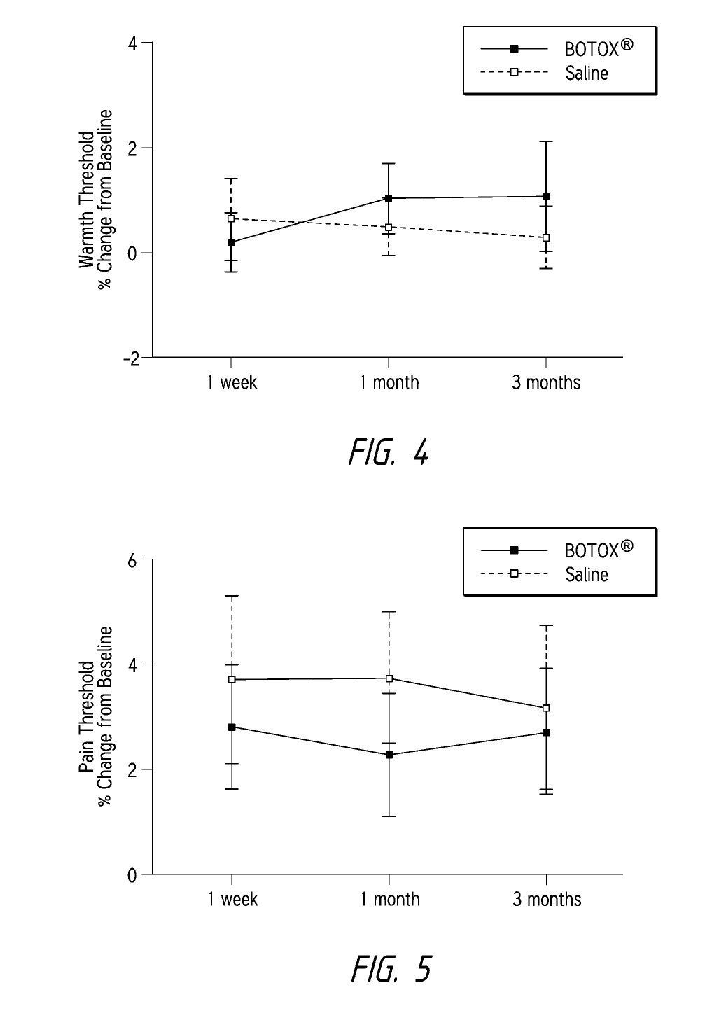

As shown in FIG. 4, the warmth threshold or thermal sensitivity change from baseline was not significantly different between the saline control and the BOTOX® treatment.

Similarly, as shown in FIG. 5, the pain threshold change from baseline was not significantly different between the saline control and the BOTOX® treatment

In conclusion, in this study, it was found that BOTOX® caused a significant reduction in itch VAS intensity for up to 3 months' post-treatment (FIGS. 2A-2E and 3A-3D) without affecting thermal sensitivity (FIG. 4) or pain thresholds (FIG. 5). Taken together, this data suggests that BOTOX® targets a specific subset of neurons that mediate itch, without affecting neurons that mediate heat and pain sensation.

Many alterations and modifications may be made by those having ordinary skill in the art, without departing from the spirit and scope of the disclosure. Therefore, it must be understood that the described embodiments have been set forth only for the purposes of examples, and that the embodiments should not be taken as limiting the scope of the following claims. The following claims are, therefore, to be read to include not only the combination of elements which are literally set forth, but all equivalent elements for performing substantially the same function in substantially the same way to obtain substantially the same result. The claims are thus to be understood to include those that have been described above, those that are conceptually equivalent, and those that incorporate the ideas of the disclosure.

1. A method for treating a histamine-independent pruritic itch in a patient in need thereof, comprising locally administering a composition comprising a therapeutically effective amount of a clostridial toxin derivative to an itch affected area.

2. The method of claim 1, whether the administering is by injection.

3. The method of claim 1, wherein the administering is topically.

4. The method of claim 1, wherein the clostridial toxin derivative is a botulinum toxin.

5. The method of claim 4, wherein the botulinum toxin is selected from the group consisting of botulinum toxin types A, B, C, D, E, F and G.

6. The method of claim 5, wherein the botulinum neurotoxin is type A.

7. The method of claim 8, wherein the therapeutically effective amount is between about 1 Units to about 500 U per patient, preferably in the range of 10-460 U per patient.

8. The method of claim 1, wherein the patient is suffering from a pruritic condition comprising atopic dermatitis, nervous dermatitis, contact dermatitis, seborrheic dermatitis, autosensitization dermatitis, caterpillar dermatitis, asteatosis, senile pruritus cutaneous, insect sting, photosensitive dermatosis, urticaria, prurigo, herpes, impetigo, eczema, tinea, lichen, psoriasis, scabies and acne vulgaris, or visceral diseases complicated with pruritus.

9. The method of claim 8, wherein said visceral diseases complicated with pruritus comprise malignant tumors, diabetes mellitus, hepatic diseases, renal failure or pregnancy

10. The method of claim 1, wherein the patient is suffering from a skin change comprising pruritus secondary to inflamed skin, pruritus arising from conditions of non-diseased skin, pruritus associated with chronic secondary scratch, or skin lesions resulting from an underlying medical condition.

11. The method of claim 10, wherein said underlying medical condition has an origin comprising dermatologic origin, systemic disease origin, neurologic origin, psychogenic origin, or mixed origin.

12. The method of claim 1, wherein said subject has uremic pruritus or prurigo nodularis.

13. The method of claim 1, further comprising administering a second antipruritic therapy.

14. The method of claim 13, wherein the second antipruritic therapy comprises an anti-histamine therapy.

15. The method of claim 13, wherein the antipruritic therapy comprises opioids, NK1 antagonists, TRPV antagonists, TRPA antagonist, and PAR2 antagonists.

16. The method of claim 13, wherein the second antipruritic therapy comprises a therapeutic agent suitable for treatment of skin diseases.

17. The method of claim 16, wherein the therapeutic agent comprises steroids, calcineurin inhibitors, anti-TNF, anti-IL-17, anti-IL-23, anti-IL-31, anti-TSLP, anti-IL4R treatments, retinoids, vitD analogs, PDE4 inhibitors, or combinations thereof.

18. The method of claim 1, wherein the administrating alleviates itch intensity or duration without affecting a sensation of pain or heat.

19. A method for alleviating a symptom associated with histamine-independent itch treating a histamine-independent pruritic itch, comprising locally administering a composition comprising a therapeutically effective amount of a clostridial toxin derivative to an itch affected area.

20. The method of claim 19, wherein the symptom comprises pain, discomfort, inflammation or combinations thereof.

21. The method of claim 19, wherein the administering alleviates itch intensity or duration without affecting a sensation of pain or heat.

22. The method of claim 19, whether the administering is by injection.

23. The method of claim 19, wherein the administering is topically.

24. The method of claim 19, wherein the clostridial toxin derivative is a botulinum toxin.

25. The method of claim 24, wherein the botulinum toxin is selected from the group consisting of botulinum toxin types A, B, C, D, E, F and G.

26. The method of claim 25, wherein the botulinum neurotoxin is type A.

27. A method for reducing the occurrence of a histamine-independent pruritic itch, comprising locally administering a composition comprising a therapeutically effective amount of a clostridial toxin derivative to an itch affected area.

28. The method of claim 27, whether the administering is by injection.

29. The method of claim 27, wherein the administering is topically.

30. The method of claim 27, wherein the clostridial toxin derivative is a botulinum toxin.

31. The method of claim 31, wherein the botulinum toxin is selected from the group consisting of botulinum toxin types A, B, C, D, E, F and G.

32. The method of claim 31, wherein the botulinum neurotoxin is type A.

33. The method of claim 27, wherein the administering alleviates itch intensity or duration without affecting a sensation of pain or heat.
